(19) United States
(12) Patent Application Publication
(10) Pub. No.: US 2019/0211303 A1
(43) Pub. Date: Jul. 11, 2019

(54) A METHOD OF PRODUCING MIXED MICROBIAL CULTURES

(71) Applicant: NIP B.V., Ede (NL)

(72) Inventors: Thom HUPPERTZ, Ede (NL); Franklin Delano ZOET, Giesbeek (NL); Margaretha Maria Marke BEERTHUIJZEN, Arnhem (NL); Herwig DE HEER H. BACHMANN, Amsterdam (NL)

(73) Assignee: NIP B.V., Ede (NL)

(21) Appl. No.: 16/328,213

(22) PCT Filed: Aug. 23, 2017
(86) PCT No.: PCT/EP2017/071242

§ 371 Date: Feb. 25, 2019

(30) Foreign Application Priority Data
Aug. 26, 2016 (EP) 16185962.4

(51) Int. Cl. C12N 1/20; A23L 33/135; A23D 7/005; A23K 10/18; A23L 2/52

(52) U.S. Cl. CPC C12N 1/20; A23L 33/135; A23D 7/005; A23V 2002/00; A23L 2/52; C12N 2502/70; A23K 10/18

(57) ABSTRACT

The invention relates to a method of propagating a mixture of two or more different micro-organism phenotypes, said method comprising the steps of: a) inoculating an aqueous culture medium with an inoculum comprising at least two different micro-organism phenotypes; b) mixing the inoculated aqueous medium with fat to produce a water-in-oil emulsion; c) incubating the emulsion at an incubation temperature in the range of 20-60° C. for at least 2 hours; d) heating the incubated emulsion to a temperature that is at least 5° C. above the incubation temperature to cause phase separation of the emulsion; e) repeating the cycle of steps a) to d) at a larger scale using viable cells contained in the aqueous phase of the phase separated emulsion as the inoculum; and f) collecting the propagated mixture of the two or more different micro-organism phenotypes wherein the fat has a solid fat content at the incubation temperature (N_{Tc}) of at least 5 wt. %. The method according to the invention enables industrial scale production of mixed microbial cultures starting from an inoculum containing a mixture of micro-organisms with no, or only minor population variation during propagation, even if the inoculum contains both fast and slow growing micro-organisms.

### TECHNICAL FIELD OF THE INVENTION

The present invention relates to a method of producing mixed microbial cultures by propagating a mixture of micro-organisms. More particularly, the present invention relates to such a production method that employs propagation in emulsified growth medium.

The present method offers the advantage that it enables the production of mixed cultures at an industrial scale starting from an inoculum that contains a mixture of micro-organisms, with no more than minor changes in the microbial population during propagation.

### BACKGROUND OF THE INVENTION

Complex mixtures of micro-organisms are of unquestionable importance for many natural and industrial processes. In nature, microbial consortia are found, for instance, in soil and in the digestive tract of animals and humans, where they play an important role in the biodegradation of a wide variety of substrates. Complex mixture of micro-organisms are also used industrially, e.g. in the production of several types of traditional food products (cheese, sausages etc.) and in the production of probiotic foods.

Probiotics, i.e. microorganisms that are believed to provide health benefits when consumed, can be composed of a mixture of micro-organisms (microbial consortia). It is important that these mixtures can be produced on an industrial scale in a reproducible manner.

The production of mixed microbial cultures in liquid growth medium is greatly challenged by microbial competition among the diverse populations in the culture medium. When fast-growing variants increase in the population, slower growing cells are outcompeted. This is inherently problematic in suspension culturing systems. In suspension culturing systems, unlike in agar cultures, the cells are not physically isolated in their colonies. The close proximity of cells and the free access to substrates in the medium enables the growth of fast growing micro-organisms that significantly reduces the offspring of slower growing micro-organisms. Consequently, prolonged propagation of microorganisms in suspension results in the selection of fast growing micro-organisms.

One way of reproducibly producing mixed microbial cultures is to separately propagate the different strains and to afterwards combine the propagated strains in the desired ratio. This approach, however, is undesirably laborious and costly if the mixed microbial culture contains a large number of different strains. Furthermore, for undefined cultures the individual strains are not available/known and therefore separate culturing is not an option.

Emulsion propagation has been used as a tool for selecting metabolically efficient micro-organisms.

WO 2012/093128 describes a method of selecting a cell metabolically efficient under conditions of high substrate concentration expressing a desired phenotype by serial propagation in a water-in-oil emulsion-based system. In Example 1, a diluted culture containing two different L. lactis strains was mixed with mineral oil and Abil90 (surfactant) to prepare a water-in-oil emulsion containing 10 vol. % dispersed aqueous phase (average droplet size 35-40 μm) and 90 vol. % continuous oil phase. It was shown that a strain with a desired phenotype, like a high yield strain, is selectively enriched or stabilized in the emulsion-based system disclosed therein, when compared to suspension cultures.

Bachmann et al. (Availability of public goods shapes the evolution of competing metabolic strategies, PNAS | Aug. 27, 2013 | vol. 110 | no. 35 | 14303) investigated serial propagation of a microbial population in a water-in-oil emulsion in order to select strains with increased biomass yield. The following test conditions are described in the article: Three hundred microliters of a freshly inoculated L. lactis culture was used to make an emulsion by shaking it for 3-4 min with 700 μL HFE7500 (3M Novec) on a vortex mixer. The oil was supplemented with 0.5% vol/vol surfactant. Shaking was carried out at 2,200-3,200 rpm in capped 10-mL tubes. After shaking an emulsion separated in the tube from the surplus of oil within a few minutes and 650 μL of the oil phase was removed from the bottom of the tube. Cells were allowed to grow in emulsion at 30° C. for 1 or 2 d and subsequently the emulsion was broken. The breaking was done by adding 300 μL of 1H,1H,2H,2H-perfluorooctanol and frequent light shaking of the tube over a period of up to 15 min. The results demonstrated the impact of privatizing a public good on the evolutionary outcome between competing metabolic strategies, and that the serial propagation of a microbial population in a water-in-oil emulsion allows selection of strains with increased biomass yield.

WO 2009/115660 relates to a method for in vivo selection of live microorganisms on the basis of the enzyme activity expressed by said cells outside the cytoplasm of their cytoplasm. The method comprises the following steps:
a) preparing an aqueous phase containing a diverse population of cells expressing extracellular enzymes and a substrate of these enzymes enabling selection of said cells on the basis of the activity of these enzymes;
b) separately preparing an oil phase containing oil and surfactants;
c) preparing an emulsion by dispersing the aqueous phase into the oil phase;
d) allowing the cells to grow within the aqueous droplets of the emulsion.

WO 2016/018678 discloses a method of detection of bacteriophages. The detection method comprises:
creating a water-in-oil (W/O) emulsion, comprising:
   suspending a bacterial cell mixture in an inner aqueous phase (W1) comprising a water soluble emulsifier and a cell viability dye, wherein the bacterial cell mixture comprises the sample suspected of comprising bacteriophage; and
   suspending droplets of the inner aqueous phase (W1) into an oil phase (O) comprising an oil and a hydrophobic emulsifier having an HLB value of 4 or less, thereby yielding a water-in-oil (W1/0) emulsion; and
detecting the cell viability dye, wherein detectable cell viability dye provides a signal when bacterial cells within the water-in-oil (W1/0) emulsion are non-viable, thereby indicating the presence of bacteriophage in the sample suspected of comprising bacteriophage.

### SUMMARY OF THE INVENTION

The inventors have developed a method of reproducibly producing mixed microbial cultures at an industrial scale by propagating a mixture of micro-organisms. More particularly, the method of the invention relates to a method of propagating a mixture of two or more different micro-organism phenotypes, said method comprising the steps of:
a) inoculating an aqueous culture medium with an inoculum comprising at least two different micro-organism phenotypes to produce an inoculated aqueous medium containing 10²-10⁷ viable cells/ml;
b) mixing the inoculated aqueous medium with fat to produce a water-in-oil emulsion having a volume weighted average droplet size of 10-2000 μm;
c) incubating the emulsion at an incubation temperature (Tc) in the range of 5-60° C. for at least 2 hours;
d) heating the incubated emulsion to a temperature that is at least 5° C. above the incubation temperature to cause phase separation of the emulsion;
e) repeating the cycle of steps a) to d) at a larger scale using viable cells contained in the aqueous phase of the phase separated emulsion as the inoculum; and
f) collecting the propagated mixture of the two or more different micro-organism phenotypes

wherein the fat contains at least 90 wt. % of glycerides selected from triglycerides, diglycerides and combinations thereof; and wherein the fat has a solid fat content at the incubation temperature (N_{Tc}) of at least 5 wt. %.

The method according to the invention enables industrial scale production of mixed microbial cultures starting from an inoculum containing a mixture of micro-organisms with no or only minor population variation during propagation, even if the inoculum contains both fast and slow growing micro-organisms The present method is perfectly suited for producing mixed microbial cultures that contain a large number of different strains and for reproducibly producing undefined mixed cultures. In addition, the present method can be carried out using only food grade materials.

Although the inventors do not wish to be bound by theory, it is believed that the present method allows mixtures of micro-organisms to be propagated without substantial changes in microbial population because the different strains are allowed to grow in isolation in separated micro-environments, i.e. droplets of aqueous phase. Thus, there is essentially no competition between these strains during incubation/propagation. The concentration of the micro-organisms in the inoculated culture medium and the size of the aqueous phase droplets in the water-and-oil emulsion are important factors in the present method as they determine the occupation of the aqueous phase droplets. Ideally, the emulsion volume is prepared in such a way that each droplet is inoculated with exactly one cell, to prevent cell-cell competition. In practice, this cannot be achieved as the distribution of cells over the water droplets follows a Poisson distribution. However, cell-cell competition can effectively be avoided, e.g. by preparing an emulsion in which 1 in 10 droplets is occupied with a single cell. Droplet occupation follows a Poisson distribution as described by Bachmann et al. (PNAS | Aug. 27, 2013 | vol. 110 | no. 35 | 14303). To increase total biomass yield, emulsion droplet occupation can be increased, but that will also increase multiple occupations of droplets. The optimum droplet occupation will depend on the type of microbial consortium to be propagated.

The present method is easy to operate because it employs a water-in-oil emulsion that is stable under the conditions employed during incubation, but that can easily be phase separated by simple heating. Thus, the aqueous phase containing the propagated mixture of micro-organisms and the fat phase can easily be isolated from the emulsion after heat-induced phase separation. The cycle comprising formation of the inoculated water-in-oil emulsion; incubation; and phase separation can be repeated multiple times at an increasing scale so as to increase the yield of propagated micro-organisms. Once the desired yield has been achieved, the aqueous phase containing the propagated micro-organisms can be isolated from the fat phase and the mixture of micro-organisms can be collected. The isolated fat phase may be reused in the present method.

The present invention further relates to a propagated mixture of micro-organisms obtained by the present method and to a process of preparing an edible product by combining one or more edible ingredients with said propagated micro-organism mixture.

### DETAILED DESCRIPTION OF THE INVENTION

A first aspect of the invention relates to a method of propagating a mixture of two or more different micro-organism phenotypes, said method comprising the steps of:
a) inoculating an aqueous culture medium with an inoculum comprising at least two different micro-organism phenotypes to produce an inoculated aqueous medium containing 10²-10⁷ viable cells/ml;
b) mixing the inoculated aqueous medium with fat to produce a water-in-oil emulsion having a volume weighted average droplet size of 10-2000 μm;
c) incubating the emulsion at an incubation temperature (Tc) in the range of 5−60° C. for at least 2 hours;
d) heating the emulsion to a temperature that is at least 5° C. above the incubation temperature to cause phase separation of the emulsion;
e) repeating the cycle of steps a) to d) at a larger scale using viable cells contained in the aqueous phase of the phase separated emulsion as the inoculum; and
f) collecting the propagated mixture of the two or more different micro-organism phenotypes

wherein the fat contains at least 90 wt. % of glycerides selected from triglycerides, diglycerides and combinations thereof; and wherein the fat has a solid fat content at the incubation temperature (N_{Tc}) of at least 5 wt. %, said solid fat content being determined by the ISO 8292-1 (2012) method.

The term “micro-organisms phenotypes” as used herein refers to the expression of a genotype (i.e. the full genetic complement) of a micro-organism in a given environment. Within an individual organism, both changes in genetic makeup, such as from bacterial conjugation, and variation in gene expression can result in different phenotypes under similar environmental conditions. Conversely, environmental variation can lead to different outcomes for genetically identical organisms, through variable gene expression.

The term “aqueous culture medium” as used herein refers to an aqueous growth medium that supports the growth of the micro-organisms contained in the inoculum.

The term “fat” as used herein refers to naturally occurring lipids including fatty acid glyceride esters (including phospholipids), fatty acids and waxes.

The term “phase separation” as used herein refers to the transition of the water-in-oil emulsion to a de-emulsified system in which at least a part of the originally dispersed aqueous phase is present as a separate continuous phase. Typically, phase separation of the water-in-oil emulsion in the present method leads to the formation of an aqueous bottom layer containing viable cells and a top layer containing the fat.

The volume weighted average droplet size of the dispersed aqueous phase in the water-in-oil emulsion can suitably be determined by pulsed field gradient NMR using the methodology described by Van Duynhoven et al. (Scope of droplet size measurements in food emulsions by pulsed field gradient NMR at low field. Magnetic Resonance in Chemistry, (2002), 40(13), 51-59). If the droplets of the aqueous are relatively large, the aforementioned NMR method may be less suitable, in which case the volume weighted average droplet size can be determined by means of microscopic image analysis as described by Jokela et al. (The use of computerized microscopic image analysis to determine emulsion droplet size distributions. Journal of colloid and interface science, (1999) 134(2), 417-426).

The solid fat content of the fat at a given temperature can suitably be determined using the method described in ISO 8292-1 (2012)—Determination of solid fat content by pulsed NMR.

The propagation method of the present invention may suitably be carried out under aerobic or anaerobic conditions.

The inoculum employed in the present method comprises two or more different micro-organism phenotypes. The micro-organisms that can be employed include prokaryote as well as eukaryote. Preferably, the micro-organisms are selected from bacteria and fungi (including yeast). More preferably, the micro-organisms are selected from bacteria, most preferably from lactic acid bacteria, Bifidobacteria, and combinations thereof.

The micro-organisms that are propagated using the present method can be sampled from, for instance, complex cultures for food or feed fermentation, mixed cultures for bioprotection, complex probiotics, from microbiota (e.g. skin, gut, oral cavity, vagina, nose), etc. The present method may also be used to produce complex mixtures of micro-organisms that can be used for microbiota transplantations, e.g. fecal microbiota transplantation. Fecal microbiota transplantation (FMT) is the process of transplanting fecal bacteria from a healthy individual into a recipient. FMT involves restoration of the colonic microflora by introducing healthy (pathogen-free) bacterial flora, e.g. by enema, orogastric tube or by mouth. Currently, FMT usually comprises infusion of stool obtained from a healthy donor. The present method enables propagation of (fecal) microbiota whilst largely maintaining the original population thus reducing the need for donor material and avoiding the infusion of stool.

The benefits of the present method are particularly appreciated in case the inoculum contains at least 3 different micro-organism phenotypes. More preferably, the inoculum contains at least 4, most preferably at least 5 different micro-organism phenotypes.

In accordance with another preferred embodiment, the inoculum contains at least 3, more preferably at least 4 and most preferably at least 5 different micro-organism strains.

According to another preferred embodiment, the micro-organism phenotype that is most abundant in the inoculum in terms of plate count represents not more than 99.99%, more preferably not more than 99.9% and most preferably not more than 99% of the inoculum, said percentage being calculated on the basis of plate count.

The aqueous culture medium employed in the present method typically contains at least 70 wt. % water. More preferably, the aqueous culture medium contains at least 80 wt. %, most preferably 90 wt. % water. Besides water, the aqueous culture medium contains a carbon and nitrogen source and optionally any other ingredients needed by the organisms to grow, such as salts providing essential elements such as magnesium, phosphorus and sulfur.

The inoculated aqueous medium preferably contains 10³-5×10⁷, most preferably 10⁴-10⁶ viable cells/ml.

The fat employed in the present method preferably has a solid fat content at 20° C. (N₂₀) of at least 10%, more preferably of at least 15%, most preferably of at least 20%.

According to a particularly preferred embodiment, the fat that is employed in the present method to prepare the water-in-oil emulsion preferably contains a significant amount of solid fat at the temperature at which the emulsion is incubated. The solid fat stabilizes the water-in-oil emulsion and prevents coalescence and gravitational separation of the dispersed aqueous phase droplets. Preferably, the fat has a solid fat content at the incubation temperature (N_{Tc}) of at least 8 wt. %, more preferably of at least 10 wt. %, even more preferably of at least 12 wt. % and most preferably of 15-50 wt. %.

The fat used in the propagation method of the invention typically contains at least 90 wt. %, preferably at least 95 wt. %, most preferably at least 98 wt. %, of glycerides selected from triglycerides, diglycerides and combinations thereof.

The fat used in the present method preferably is an edible fat, more preferably an edible fat of vegetable origin. Examples of fats of vegetable origin include vegetable oils, fractions of vegetable oils, interesterified vegetable oils, hydrogenated vegetable oils and combinations thereof.

The solid fat that is present in the fat at the incubation temperature preferably disappears quickly when the fat is heated to a higher temperature. According to a particularly preferred embodiment, at a temperature that lies 10° C. above the incubation temperature, the fat contains less than 8% solid fat (N_{Tc+10}<5%). More preferably, said solid fat content is less than 5%, more preferably less than 3%.

In step b) of the present method, emulsification by mixing may be carried out by any means familiar to those skilled in the art. Preferably, emulsification is carried out at a temperature below 50° C., more preferably at a temperature below 40° C. and most preferably at a temperature of 35° C. The skilled person will readily establish the proper emulsification temperature, depending on the heat stability of the micro-organisms that need to be propagated.

In a preferred embodiment, the water-in-oil emulsion is prepared by mixing the inoculated aqueous medium with the fat at moderately high temperature, followed by cooling to increase the solid fat content and to thereby stabilize the emulsion. Deep cooling (e.g., to 20° C. or lower) may be used to achieve rapid stabilization of the emulsion.

Typically, the water-in-oil emulsion contains 5-70 wt. % of dispersed aqueous phase and 30-95 wt. % of continuous fat phase, more preferably 10-50 wt. % of dispersed aqueous phase and 50-90 wt. % of continuous fat phase, most preferably 15-45 wt. % of dispersed aqueous phase and 55-85 wt. % of continuous fat phase.

In a preferred embodiment of the invention, at least one emulsifier is employed in the preparation of the water-in-oil emulsion. Typically, the one or more emulsifiers are present in a concentration of 0.05-3%, preferably 1-2.5%, by weight of the water-in-oil emulsion. Suitable emulsifiers include monoglycerides, phospholipids, protein, acid esters of monoglycerides, acid esters of diglycerides, sorbitan esters, sucrose esters, polysorbates, polyglycerol esters, propylene glycol fatty acid esters, fatty acid lactylates, and combinations thereof.

Preferably, the one or more emulsifiers are employed have a low hydrophilic-lipophilic balance (HLB) value, preferably an HLB value of not more than 7, more preferably in the range of 3 to 6.

In a further preferred embodiment, hydrocolloids are introduced in the aqueous culture medium in order to stabilize the water-in-oil emulsion, e.g. in a concentration of 0.05-5%, more preferably of 0.1-2% by weight of water. Suitable hydrocolloids include gelling agents and thickening agents.

According to a particularly preferred embodiment, the dispersed aqueous phase of the water-in-oil emulsion has a high level of monodispersity, i.e. a narrow droplet-size distribution. Typically, the droplet-size distribution of the water-in-oil emulsion is such that D_{SD}/Dₘₑₐₙ 1.0 wherein: D_{SD} is the standard deviation of the droplet size; and Dₘₑₐₙ is the volume weighted average droplet size. More preferably, D_{SD}/Dₘₑₐₙ≤0.8 and most preferably D_{SD}/Dₘₑₐₙ≤0.5.

Preferably, the volume weighted average droplet size of the water-in-oil emulsion is in the range of 15-500 μm, more preferably in the range of 30-300 μm, most preferably in the range of 50-200 μm. The use of emulsions containing a dispersed aqueous phase having a relatively large droplet-size offers the advantage that high propagation yields can be achieved in a single propagation step with little shift in microbial population.

According to a particularly preferred embodiment, the number of viable cells introduced in the water-in-oil emulsion at the start of incubation is in the range of 0.01-2 per droplet of dispersed aqueous phase, wherein the number of said droplets of aqueous phase is calculated by dividing the volume of aqueous phase by the volume weighted average droplet size of the dispersed aqueous phase. More preferably, the number of viable cells in water-in-oil emulsion is in the range of 0.05-1 per droplet, even more preferably in the range of 0.08-8 per droplet, most preferably in the range of 0.1-0.5 per droplet.

The following table shows how the aforementioned parameter is calculated.

| | 1 | 2 | 3 |
|---|---|---|---|
| Viable cells/ml of aqueous phase | 10⁸ | 10⁵ | 10⁶ |
| Vol. % aqueous phase in W/O emulsion | 40 | 40 | 40 |
| Volume weighted average droplet size | 20 | 200 | 80 |
| (diameter in μm) | | | |
| Number of droplets/ml aqueous phase¹ | 2.4 × 10⁸ | 2.4 × 10⁵ | 3.7 × 10⁶ |
| Nr. of viable cells per droplet | 0.4 | 0.4 | 0.3 |

¹assuming that the droplets are perfect spheres (volume = 4/3 πr³)

The incubation temperature (Tc) in step c) of the present method is preferably in the range of 12-55° C., more preferably 15-52° C. and most preferably 18-50° C. Typically, the incubation period is in the range of 3 hours to 5 days, more preferably of 8 hours to 3 days. The incubation temperature and time will depend on the inactivation temperature and growth rate of the micro-organisms inoculated therein.

In the present method, after incubation, the emulsion is heated to a higher temperature to cause phase separation and to enable reuse of the phase separated emulsion as inoculant in step a) of the method or to enable isolation of the aqueous phase containing viable cells. The emulsion is preferably heated to a temperature at least 7° C., more preferably at least 10° C., above the incubation temperature.

After the incubated emulsion has been phase separated, the aqueous phase of the separated emulsion or the complete separated emulsion can be combined with aqueous culture medium and diluted to start a new propagation cycle (step (a)).

Once the present method has yielded the desired amount of propagated micro-organisms (at the largest scale of propagation), the propagated mixture of the two or more different micro-organism phenotypes is collected. Preferably, collection of the mixture of micro-organism phenotypes comprises isolation of the aqueous phase containing viable cells after phase separation of the emulsion. Isolation of the aqueous phase may suitably be achieved by means of decanting and/or centrifugation.

The present method is suitably carried out on a semi-industrial or industrial scale. According to a preferred embodiment, in the final cycle of steps a) to e), step c) comprises incubating at least 10 l, preferably at least 100 l, of the water-in-oil emulsion.

The method of propagating mixed cells according to the invention is advantageously stable when compared, e.g., to propagation in suspension medium. In evolutionary ecology, the Shannon's diversity index is used to assess the diversity of cultured populations comprising i different species:

$${H}^{′} = - {\sum }_{i = 1}^{R} {p}_{i} ln {p}_{i}$$

wherein:

pᵢ is the proportion of individuals belonging to the ith species in the dataset of interest. The bigger the Shannon index the larger the diversity.

The present method makes it possible to propagate mixtures of micro-organisms without introducing a major change in the diversity of the microbial population. Accordingly, it is preferred that the Shannon index of the microbial population does not change substantially. This can be expressed by the following equation:

[(H′₀−H′ₜ)]/H′₀<0.8

wherein:

H′₀ represents the Shannon index of the microbial population in the aqueous culture medium; and

H′ₜ represents the Shannon index of the collected propagated mixture. More preferably, [(H′₀−H′ₜ)]/H′₀<0.6, even more preferably [(H′₀−H′ₜ)]/H′₀<0.4 and most preferably [(H′₀−H′ₜ)]/H′₀<0.1.

The incubation step c) of the present method preferably induces a substantial growth of the micro-organisms contained in the water-in-oil emulsion. Preferably, the aqueous phase of the phase separated emulsion contains at least 10 times more viable cells than the inoculated aqueous medium. Preferably, said separated aqueous phase contains at least 20 times, more preferably at least 50 times and, even more preferably at least 80 times more viable cells than the inoculated aqueous medium.

A second aspect of the invention relates to a propagated mixture of micro-organisms obtained by the method according to the invention.

A third aspect of the invention relates to a process of preparing a product selected from food products, beverages, nutritional products and animal feed, said process comprising one or more edible ingredients with a propagated micro-organism mixture according to the invention. Examples of food products in which the propagated micro-organism mixture can be applied include fermented milk products (e.g. cheese, yogurt, kefir), fermented meat (e.g. sausages), fermented soy products (e.g. kecap, fermented soy paste), bread and probiotic food products. Examples of beverages in which the propagated mixture can be applied include fermented diary drinks, fermented soy drinks, wine, beer and distilled beverages. The propagated mixtures can also be applied in animal feed products such as silage and probiotic feed.

A fourth aspect of the invention relates to the use of the propagated micro-organism mixture as a phytoprotective agent, said use comprising applying the propagated micro-organisms mixture onto plants or plant parts. The microbial mixture may suitably be applied onto the seeds, leaves, stems or flowers of plants, e.g. by spraying or brushing.

A yet further aspect of the invention relates to the use of emulsion propagation in the production of a mixture of at least two viable micro-organism phenotypes, wherein the emulsion propagation comprises incubating a water-in-oil emulsion comprising:
a continuous fat phase having a solid fat content at 20° C. (N₂₀) of at least 10%; and
a dispersed aqueous phase having a volume weighted average droplet size of 10-250 μm, said dispersed aqueous phase comprising the at least two viable micro-organism phenotypes.

Preferred embodiments of this particular use of emulsion propagation have been described herein before in relation to the present propagation method.

The invention is further illustrated by means of the following non-limiting examples.

### EXAMPLES

### Example 1

Emulsions were prepared on the basis of the formulations shown in Table 1.

TABLE 1

| | Parts by weight | |
|---|---|---|
| | emulsion | emulsion |
| Components | 1A | 1B |
| Hardstock fat¹ | 9.36 | 10.36 |
| Sunflower seed oil | 42.64 | 41.64 |
| water + coloring agent | 11.40 | 11.40 |
| Polyglycerol polyricinoleate | 1.60 | 1.60 |
| (PGPR) | | |

¹Delico ® 474, ex Unimills, the Netherlands

The emulsions were prepared by melting the hardstock fat at 47.5° C. for 60 minutes, and admixing the sunflower oil and the emulsifier (PGPR). The fat blend was subsequently cooled down to 37° C. for 60 minutes. At 37° C., the water phase (also at 37° C.) was added to the fat blend in a 60 ml glass tube. The glass tubes were shaken by hand for 60 seconds and immediately cooled down to 5° C. (for 30 minutes).

The emulsions obtained were solid at 5° C. The majority of the droplets in emulsion 1A had a diameter in range of 50 to 200 μm. The majority of the droplets in emulsion 1B had a diameter in the range of 20 to 100 μm. The droplet size distributions of both emulsions allow for significant bacterial growth.

The stability of the two emulsions under propagation conditions was tested by incubating the emulsions at 23° C. for 18 hours. Both emulsions were found to be stable throughout the incubation period.

Subsequently, both emulsions were heated to 37° C. for 60 minutes. The emulsions became liquid and separated into a aqueous layer and an oil layer.

### Example 2

The preparation of emulsion 1B as described in Example 1 was repeated, except that this time the water phase and fat blend were mixed with an Ultra Turrax (IKA) for 20 seconds and immediately cooled down to 5° C. (for 30 minutes). The emulsion (Emulsion 2) so obtained was solid at 20° C.

The average droplet size of the dispersed aqueous phase was less than 20 μm. This droplet size distribution also allows bacterial growth, but cell growth in such relatively small water droplets is only useful for cell/medium combinations that generate high cell densities upon propagation.

Like emulsions 1A and 1B, also emulsion 2 was stable when incubated at 23° C. for 18 hours. Emulsion 2 also separated into an aqueous layer and an oil layer when heated to 37° C. for 60 minutes.

### Example 3

Different propagation emulsions were prepared using a fat phase that contained hardstock, sunflower oil and PGPR in the same ratios as the fat phase of emulsion 1B of Example 1. The propagation emulsions were prepared by mixing and cooling the fat phase with a lactococcal growth medium (M17 broth—Oxoid Cat. #CM0817 supplemented with 0.5% w/v glucose) in a glass tube as described in Example 1. The emulsions were prepared using different weight ratios of fat phase and growth medium, as shown in Table 2.

TABLE 2

| | Weight ratio |
|---|---|
| Emulsion | fat phase:growth medium |
| 3A | 5:1 |
| 3B | 4:2 |
| 3C | 3:3 |

In all cases a water-in-oil emulsion was obtained and the emulsions were stable at room temperature.

### Example 4

A propagation emulsion was prepared in the same way as emulsion 1B of Example 1, except that this time the aqueous phase contained lactococcal growth medium M17 (Oxoid), supplemented with glucose (0.5 wt. %), and two bacterial strains. The two strains were Lactococcuslactis NZ9000 and NZ9010 (Bongers et al IS981-Mediated Adaptive Evolution Recovers Lactate Production by IdhB Transcription Activation in a Lactate Dehydrogenase-Deficient Strain of Lactococcus lactis. J Bacteriol. (2003); 185: 4499-4507. doi:10.1128/JB.185.15.4499-4507). The L. lactis strains were equally represented in the inoculation liquid. The aqueous phase of the emulsion contained appr. 4×10⁴ viable cells/ml (2×10⁴ cells/ml from each strain), and the aqueous phase represented ˜17.5 vol. % of the propagation emulsion.

On the basis of the aforementioned data it can be calculated that before incubation the emulsion contained appr. 0.1 viable cells per droplet, as shown in Table 3.

TABLE 3

| Viable cells/ml of aqueous phase | 5 × 10⁴ |
|---|---|
| Vol. % aqueous phase in W/O emulsion | 17.5 |
| Volume weighted average droplet size (in μm) | 150 |
| Number of droplets/ml aqueous phase¹ | 5.66 × 10⁵ |
| Nr. of viable cells per droplet² | 0.088 |

¹assuming that the droplets are perfect spheres (volume = 4/3 πr³)
²using this value as lambda in a Poisson distribution gives the distribution of droplet occupation; In this specific case roughly 91% of the droplets are empty and ~8% of the droplets are inoculated with a single cell

Part of the aqueous phase that had been used in the preparation of the propagation emulsion was incubated at 23° C. for 2 days (suspension propagation). After propagation, the concentration of viable cells of each of the L. lactis strains was determined.

The inoculated emulsion was also incubated at 23° C. for 2 days. After incubation, the emulsion was phase separated by heating the emulsion to 37° C. for 60 minutes. A sample was taken from the separated aqueous phase. The concentration of viable cells of each of the L. lactis strains was determined. The results are shown in Table 4.

TABLE 4

| | Suspension | | Emulsion | |
|---|---|---|---|---|
| | propagation | | propagation | |
| | L. lactis | L. lactis | L. lactis | L. lactis |
| CFUs | NZ9000 | NZ9010 | NZ9000 | NZ9010 |
| Before incubation | 2E+04 | 2E+04 | 2E+04 | 2E+04 |
| After incubation | 6E+09 | 5E+06 | 8E+09 | 7E+08 |

In Table 5 the calculated Shannon indices of the microbial populations before and after incubation are shown.

TABLE 5

| | Suspension | Emulsion |
|---|---|---|
| | propagation | propagation |
| Shannon index before incubation (H′₀) | 0.673 | 0.693 |
| Shannon index after incubation (H′₁) | 0.007 | 0.280 |
| [(H′₀ − H′₁)]/H′₀ | 0.990 | 0.596 |

1-15. (canceled)

16. A method of propagating a mixture of two or more different micro-organism phenotypes, the method comprising:
(a) inoculating an aqueous culture medium with an inoculum comprising at least two different micro-organism phenotypes to produce an inoculated aqueous medium containing 102-107 viable cells/ml;
(b) mixing the inoculated aqueous medium with fat to produce a water-in-oil emulsion having a volume weighted average droplet size of 10-2000 μm, wherein the number of viable cells introduced in the water-in-oil emulsion is in the range of 0.01-2 per droplet of dispersed aqueous phase, and the number of droplets of aqueous phase is calculated by dividing the volume of aqueous phase by the volume weighted average droplet size of the dispersed aqueous phase;
(c) incubating the emulsion at an incubation temperature (Tc) in the range of 5-60° C. for at least 2 hours;
(d) heating the emulsion to a temperature that is at least 5° C. above the incubation temperature to cause phase separation of the emulsion;
(e) repeating steps (a) to (d) at a larger scale using viable cells contained in the aqueous phase of the phase separated emulsion as the inoculum; and
(f) collecting the propagated mixture of the two or more different micro-organism phenotypes;
wherein the fat contains at least 90 wt. % of glycerides selected from triglycerides, diglycerides and combinations thereof; wherein the fat has a solid fat content at the incubation temperature (NTc) of at least 5 wt. %, said solid fat content being determined by the ISO 8292-1 (2012) method; and
wherein the Shannon diversity indices of the microbial population meet the following requirement: [(H′₀−H′ₜ)]/H′₀<0.8
wherein:
(i) H′₀ represents the Shannon diversity index of the microbial population in the aqueous culture medium; and
(ii) H′ₜ represents the Shannon diversity index of the collected propagated mixture.

17. The method according to claim 16, wherein the fat has a solid fat content at the incubation temperature (N_{Tc}) of at least 8 wt. %.

18. The method according to claim 16, wherein the number of viable cells introduced in the water-in-oil emulsion at the start of step (c) is in the range of 0.1-2 per droplet of dispersed aqueous phase, the number of droplets of aqueous phase being calculated by dividing the volume of aqueous phase by the volume weighted average droplet size of the dispersed aqueous phase.

19. The method according to claim 16, wherein in the final cycle of steps (a) to (e), step (c) comprises incubating at least 10 l of the water-in-oil emulsion.

20. The method according to claim 19, wherein in the final cycle of steps (a) to (e), step (c) comprises incubating at least 100 l of the water-in-oil emulsion.

21. The method according to claim 16, wherein the volume weighted average droplet size of the water-in-oil emulsion is in the range of 15-500 μm.

22. The method according to claim 21, wherein the volume weighted average droplet size of the water-in-oil emulsion is in the range of 30-300 μm.

23. The method according to claim 16, wherein the aqueous phase of the phase separated emulsion contains at least 5 times more viable cells than the inoculated aqueous medium.

24. The method according to claim 16, wherein the water-in-oil emulsion contains 10-70 wt. % of dispersed aqueous phase and 30-90 wt. % of continuous fat phase.

25. The method according to claim 16, wherein an emulsifier is employed in the preparation of the water-in-oil emulsion in a concentration of 0.05-3% by weight of the emulsion.

26. The method according to claim 10, wherein the emulsifier has an HLB value of not more than 22.

27. The method according to claim 16, wherein the inoculum is obtained from microbiota.

28. The method according to claim 16, wherein the micro-organisms are bacteria.

29. The method according to claim 28, wherein the bacteria are selected from lactic acid bacteria, Bifidobacteria and combinations thereof.

30. A propagated mixture of micro-organisms obtained by a method according to claim 16.

31. A process of preparing a product selected from food products, beverages, nutritional products and animal feed, the process comprising combining one or more edible ingredients with a propagated micro-organism mixture according to claim 30.

32. A method of producing a mixture of at least two viable micro-organism phenotypes at an industrial scale with no or only minor population variation during propagation, wherein the method comprises incubating a water-in-oil emulsion comprising:
(a) a continuous fat phase having a solid fat content at 20° C. (N20) of at least 10%; and
(b) a dispersed aqueous phase having a volume weighted average droplet size of 10-2000 μm and comprising the at least two viable micro-organism phenotypes.
